(19) United States
(12) Patent Application Publication
(10) Pub. No.: US 2024/0090607 A1
(43) Pub. Date: Mar. 21, 2024

(54) Tourniquet Hat Assembly

(71) Applicant: John Perkinson, Laurens, SC (US)

(72) Inventor: John Perkinson, Laurens, SC (US)

(21) Appl. No.: 17/932,835

(22) Filed: Sep. 16, 2022

(51) Int. Cl. A42B 1/006; A61B 17/132

(52) U.S. Cl. CPC A42B 1/006; A61B 17/132

(57) ABSTRACT

A tourniquet hat assembly includes a hat, with an inner surface and an outer surface, and a tourniquet member having a strap with a first end and a second end, an adjustable member, and tightening means, such as a durable rod-like windlass structure or the like. The tourniquet member fits inside the lining of the inner surface of the hat and runs along the circumference of the hat. The tourniquet member preferably is removably attachable to the hat by positioning the tourniquet member within a channel or sleeve around the inner perimeter of the hat, so that it may be easily removed therefrom in an emergency. This assembly allows a user to carry a tourniquet with him or her at all times within the hat.

### BACKGROUND OF THE INVENTION

The present invention relates generally to wearable tourniquet assemblies that allow a user to easily carry a tourniquet with him or her at any time. More specifically, the present invention includes a wearable hat, such as a baseball hat or any other type of hat, with a tourniquet attached to the inner liner of the hat or with the tourniquet acting as the adjustable closure of the hat. Whenever a user needs to use the tourniquet, the user simply removes the tourniquet from the inside of the hat.

A tourniquet is generally defined as a device for stopping the flow of blood through a vein or artery, typically by compressing a limb with a cord or tight bandage. Various types of tourniquets are commonly used in emergency situations to stop the flow of blood until a victim can receive proper medical treatment. Examples of commonly used tourniquets are as follows:

Silicone Ring Auto-Transfusion Tourniquet

The silicone ring auto-transfusion tourniquet (SRT/ATT/EED), or surgical auto-transfusion tourniquet (HemaClear), is a simple to use, self-contained, mechanical tourniquet that consists of a silicone ring, stockinet, and pull straps that results in the limb being exsanguinated and occluded within seconds of application. The tourniquet can be used for limb procedures in the operating room, or in emergency medicine as a means to stabilize a patient until further treatment can be applied.

### Combat Application Tourniquet (CAT)

The combat application tourniquet is used by the U.S. and coalition militaries to provide soldiers a small and effective tourniquet in field combat situations. It is also used in the UK by NHS ambulance services, along with some UK fire and rescue services. The unit utilizes a windlass with a locking mechanism and can be self-applied. The CAT has been adopted by military and emergency personnel around the world.

The tourniquet strap comes completely apart to place around a limb, but it's a closed-loop system that has only a limited three-inch uptake. This means users must tightly secure the tourniquet strap to the limb prior to engaging the plastic windlass. Additionally, according to the manufacturer, for proper application the tourniquet strap must be through the plastic buckle differently depending on whether it's being used on an upper or lower extremity. The strap is secured on itself with Velcro, and there is one locking point for the windlass on this device.

Special Operations Forces Tourniquet (SOF-T)

The SOF-T is a true open-loop tourniquet with a solid metal windlass. Early versions have a narrow strap and a metal “alligator” clip with a locking screw to secure the strap. There are two plastic D-ring securing points for the windlass once applied.

Heretofore, many efforts have been made to provide wearable tourniquets. Some examples are set forth hereinbelow, and each of the following references are incorporated herein by reference, in their entireties:

U.S. Pat. No. 4,384,583 Tourniquet

A tourniquet constructed from a length of elastic material. The ends of the elastic material are made non-elastic, and complementary coupling components are attached thereto. The elastic material is provided with a thickening for stress absorption and control.

U.S. Pat. No. 7,604,651 Military Clothing Having Built-In Tourniquets

An article of clothing, body armor or garment having at least one limb-receiving sleeve includes a housing secured in encircling relation to the sleeve. A self-sustaining tourniquet is disposed within the housing. An opening is formed in the housing and a cover is provided to close the opening. The tourniquet is positioned fully within the housing when the cover is closed. The tourniquet is oriented in the housing so that a free end of the tourniquet is in registration with the opening. A handle is secured to the free end and is adapted to be grasped by a user when the cover is open. The diameter of the tourniquet is reduced when the handle is pulled upon. A ratchet and pawl structure prevents the tourniquet from loosening after the handle has been pulled.

U.S. Pat. No. 8,707,468 Protective Garment with Tourniquet

A protective garment including a waist portion adapted to be donned proximate the waist of a wearer and first and second leg portions extending from a waist portion to at least proximate a knee portion of the wearer and a tourniquet member attached to an inner surface of at least one of said first and second leg portions.

U.S. Pat. No. 9,456,826 Tourniquet Device for Medical Emergencies

A tourniquet device includes a tourniquet strap having an outward facing surface and an inward facing surface, the outward facing surface being covered with a plurality of hook fasteners, the inward facing surface having a section that is provided with a plurality of loop fasteners, a first end and a second end, the first end comprising a buckle, the second end having a cavity, and a securement strap configured for storage within the cavity, the securement strap including a terminal end that includes a pull tab, the securement strap is configured to be selectively placed in a stored position and a deployed position.

U.S. Pub No. US 200810243172 Garment with Affixed Tourniquet

The invention provides an article of clothing having a tourniquet affixed to the clothing at one end of the tourniquet, the tourniquet comprising a band comprised of an elastic and flexible material, the band having a width and a length, a first end and a second end, and a first face and second face, where at least a portion of the first face comprises hook-type fastener means, and where the second face comprises loop-type fastener means.

### BRIEF SUMMARY OF THE INVENTION

In accordance with one aspect of the invention, a first embodiment of a tourniquet hat assembly includes a hat with an inner surface and an outer surface, a channel or sleeve defined about an inner perimeter of the hat, and a tourniquet member having a strap with a first end and a second end, an adjustable member, and tightening means. The tourniquet member can be removably attached within the channel or sleeve of the inner surface of the hat and is positioned about the inner circumference of the hat. Another embodiment of a tourniquet hat assembly includes the tourniquet member being removably attached to the hat, such that the tourniquet member can be used as a means for adjusting the size of the hat to accommodate the size of a user's head. The tourniquet is preferably made of a durable, washable material. The tightening means is typically a durable rod-like structure (commonly referred to as a ‘windlass’ when used in context of a tourniquet) for winding the tourniquet when in use.

This tourniquet/hat assembly is ideal for off-duty paramedics, military, and other rescue personnel who might need to respond to an emergency situation requiring a tourniquet. Additionally, it may be integrated into uniforms for rescue personnel to ensure they carry a tourniquet with them at all times.

### BRIEF DESCRIPTION OF THE DRAWINGS

These and other features, aspects, and advantages of the present invention will become better understood with regard to the following description, appended claims, and accompanying drawings where:

FIG. 1 is a perspective view of one embodiment of a hat/tourniquet assembly, showing a tourniquet positioned within the inner rim portion of the hat, and further including tightening means for adjusting the diameter of both the hat and the tourniquet;

FIG. 2 is a cross-sectional view along the lines 2-2 shown in FIG. 1, wherein the tourniquet is disposed within an inner rim or channel disposed about a lower periphery of the hat;

FIG. 3 is a perspective view of one embodiment of a hat/tourniquet assembly, wherein the tourniquet is positioned within the inner rim of a hat, and further showing tightening means including a windlass that may be twisted to tighten the tourniquet and secured in position to maintain pressure of the tourniquet about a person's limb or body member;

FIG. 4 is a perspective view of one embodiment of a hat/tourniquet assembly, wherein the tourniquet is positioned within the inner rim of a hat, and further showing a removable windlass member that is removably attached to the bill of the hat, so that both the windlass and the tourniquet may be removed from the hat in an emergency situation;

FIG. 5 is a perspective view of one embodiment of a hat/tourniquet assembly, wherein the tourniquet is removably positioned about the inner periphery or rim of the hat within a sleeve or lip positioned thereon; and

FIG. 6 is a perspective view of one embodiment of a hat-tourniquet assembly, showing the tourniquet partially removed from the inner rim of the hat.

### DETAILED DESCRIPTION OF THE INVENTION

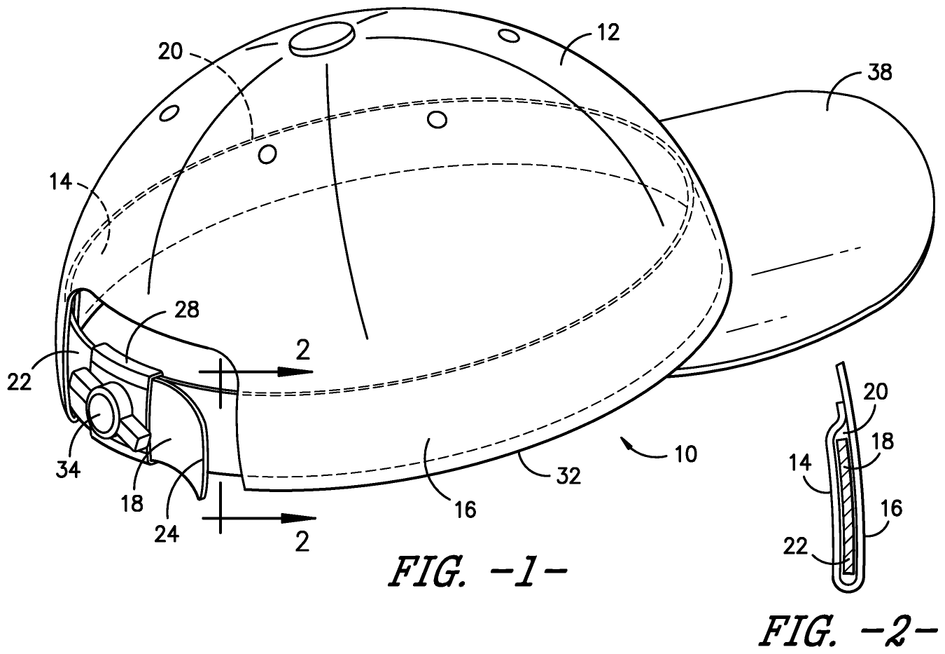

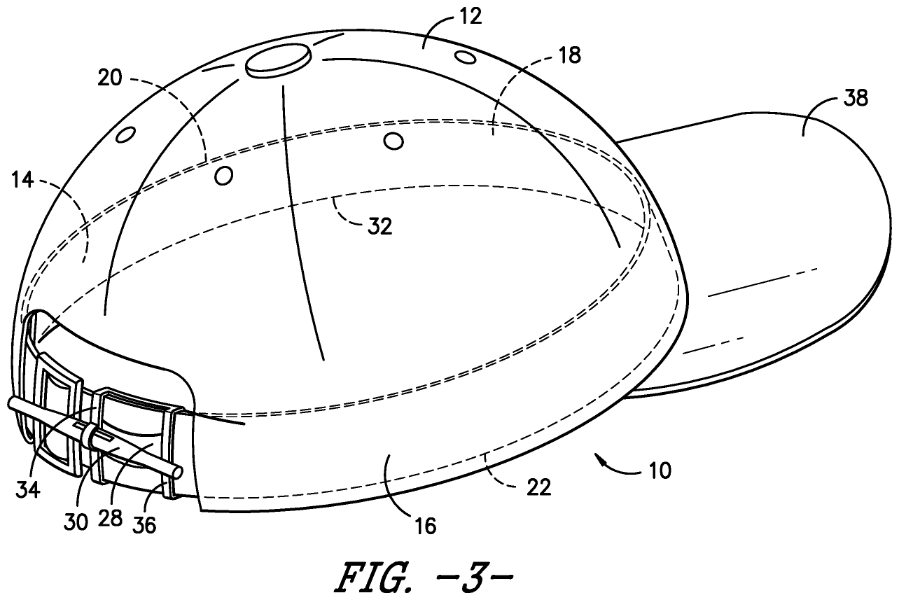

The present invention includes, in a first embodiment, a tourniquet hat assembly 10, as shown in FIGS. 1-6, and includes a baseball-style hat 12 with an inner surface 14 and an outer surface 16 and a tourniquet member 18 removably positioned within an interior portion of the hat 12, preferably within a sleeve or channel 20 about the perimeter 32 of the hat 12. The tourniquet member 18 preferably includes a strap 22 with a first end 24 and a second end 26, an adjustable member 28, and tightening means 30, such as a durable rod-like structure or the like (also referred to as a ‘windlass’ 30). The tourniquet member 18 can be made from any suitable durable material. The tourniquet member 18 preferably fits inside of a channel or sleeve 20 within the inner perimeter of the hat, as shown in FIGS. 1-6, so that a user can carry the tourniquet member 18 with him or her at all times.

In a preferred embodiment, the tourniquet member 18 fits within a channel or sleeve 20 positioned on the inner perimeter 32 of the hat 12, such that the hat 12 can be worn comfortably by the wearer while the tourniquet member 18 sits therein. Traditionally, baseball-style hats include an inner perimeter 32 that includes a channel 20, lip, sleeve, or the like, which is traditionally used to carry an adjustable strap for adjusting the size of the hat 12. In one embodiment of the present invention, the tourniquet member 18 is similarly positioned within this channel 20 or sleeve, and is removable in the event of an emergency that requires a tourniquet as part of the first aid response.

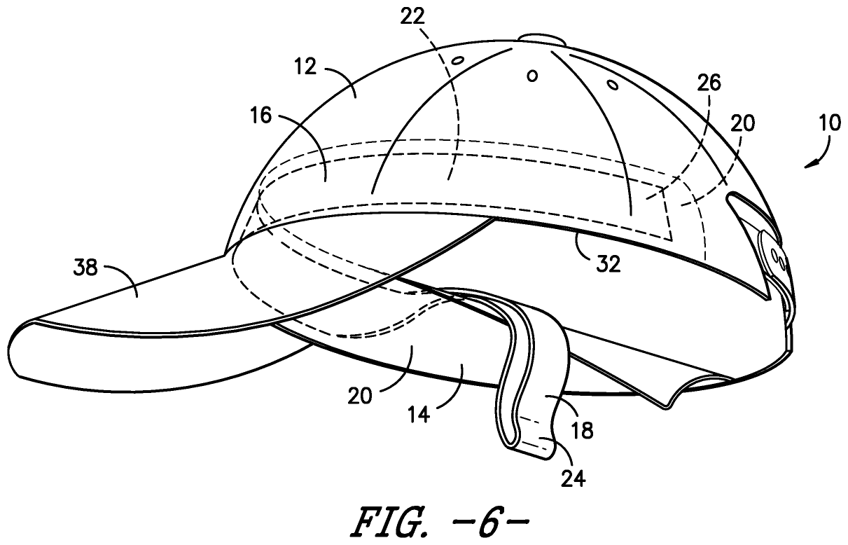

It should be understood that the hat 12 can be any style of hat, including, but not limited to a baseball cap, a fitted hat, a beanie, a bucket hat, or any other suitable style of hat. In order to secure the tourniquet member 18 to the inside of the hat 12, the tourniquet member 18 can be removably attached to the hat 12 within the channel 20 in the inner perimeter 32 thereof, as set forth above, or alternatively, with hook and loop fasteners, snaps, buttons, stitching, or other attachment means. When the need arises for the user to make use of the tourniquet member 18, the user may simply grab the first end 24 and pull the tourniquet member 18 out of the channel or sleeve 20 disposed about the inner periphery 32 of the hat 12 until it is removed therefrom, as shown in FIG. 5 (showing the tourniquet 18 in a partially removed state from the hat 12).

In one embodiment, a tightening mechanism 34 is attached to the tourniquet member 18 so that it faces away from the rear of the hat 12, as shown in FIG. 1. The tightening mechanism 34, similar to those used on snowboard boots and the like, may be tightened by twisting the handle 36 in one direction. The tightening mechanism 34 may also be loosened or released, and then readjusted, and such tightening mechanisms are well known in the art (particularly as applied to tourniquets).

Various types of tourniquets may be used, including windlass tourniquets, an example of which may be found in U.S. Pat. No. 8,343,182, which is incorporated herein by reference. For example, in one embodiment, a windlass tourniquet assembly includes an elongate main strap extending between a first end and an opposite second end, and attachment means (e.g., a buckle) for attaching the first end of the main strap to the second end of the strap. At least a portion of the attachment means is disposed at the first end of the elongate main strap. The assembly further includes a windlass strap extending between a first and second end, the first end of the windlass strap being secured to a front surface of the main strap at the first end of the main strap near the buckle or other attachment means. The second end of the windlass strap is secured to the main strap at a location a predetermined distance away from the attachment means.

Anchor means for anchoring the attachment means in a position for applying the tourniquet may be disposed on a back surface of the main strap opposite the main strap's front surface. One example of anchor means is an anchor strap including a plurality of loops formed by attaching (e.g., stitching) the anchor strap to the main strap at a plurality of locations along the length of the anchor strap. The assembly also includes an elongate windlass crank that is attachable to the anchor strap during use so that when the crank is rotated the anchor strap is twisted, effectively shortening the length of the main strap and thereby tightening the main strap (as a result of the attachment of the windlass strap to the main strap) for use as a tourniquet.

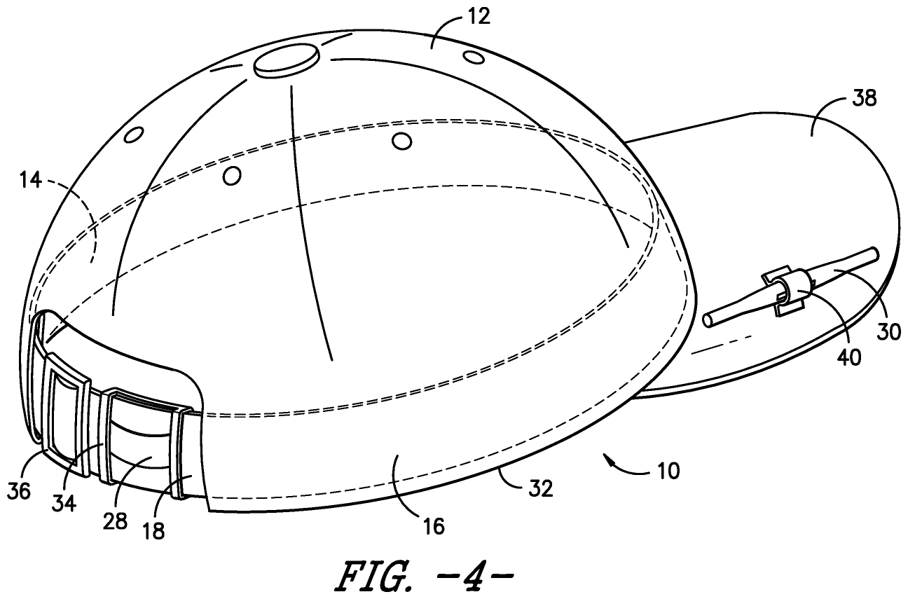

In one embodiment, the windlass crank 30 can be secured at the back portion of the hat 12 and act as an adjustable closure for the hat 12. Most baseball hats known in the art have an adjustable snapback closure or adjustable backstrap. The tourniquet member 18 fits within the hat 12 such that the windlass crank 30 of the tourniquet member 18 (or other tightening means, as shown in FIG. 1) sits at the back of the hat 12 and allows the user to adjust the size of the hat 12 by rotating the windlass crank, as shown in FIG. 3. The buckle 36 may be used to secure the tightened strap 22 in position.

It is also contemplated that the tourniquet member 18 can be removed and both the hat 12 and the tourniquet member 18 can be washed. Additionally, the hat 12 can be worn without the tourniquet member 18 if the user desires.

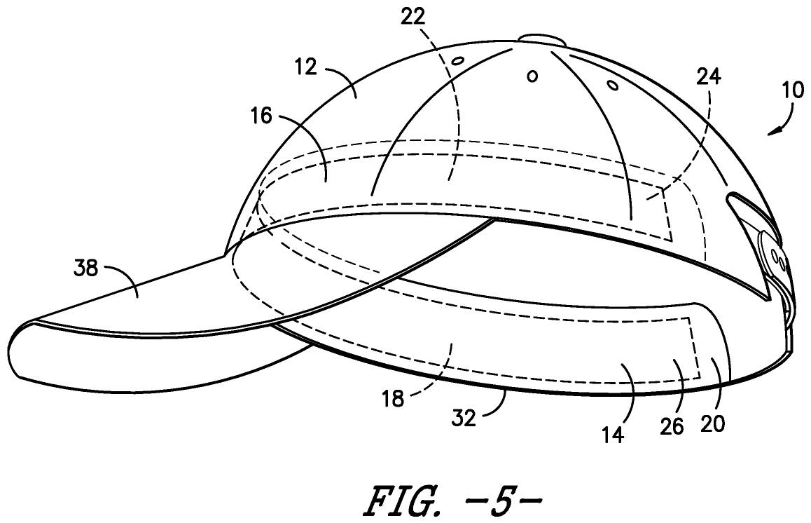

In one embodiment, as shown in FIG. 4, the windlass crank 30 is removably attached to the bill 38 of the hat 12, and may be removed therefrom, along with the tourniquet member 18, in the event of an emergency. The windlass crank 30 may be attached to the bill 38 of the hat 12 using any suitable method, including hook and loop fasteners, a rigid loop, an elastic loop 40 that stretches and frictionally engages the windlass crank 30 when it is positioned within the elastic loop 40, or any other method that allows the windlass crank 30 to be removably secured to the bill 38 of the hat 12, and further allows quick removal therefrom in the event of an emergency.

Although the present invention has been described in considerable detail with reference to certain preferred versions thereof, other versions are possible. Therefore, the spirit and scope of the appended claims should not be limited to the description of the preferred versions contained herein. All features disclosed in this specification may be replaced by alternative features serving the same, equivalent or similar purpose, unless expressly stated otherwise. Thus, unless expressly stated otherwise, each feature disclosed is one example only of a generic series of equivalent or similar features.

1. A tourniquet hat assembly, comprising:
a wearable hat having an inner surface and an outer surface and a perimeter;
a tourniquet member, having a strap with a first end and a second end, an adjustable member, and tightening means;
wherein said tourniquet member is removably positioned adjacent said inner surface of said hat.

2. The tourniquet hat assembly set forth in claim 1, wherein said hat is selected from the group consisting of a baseball hat, a bucket hat, a beanie, and a military cap.

3. The tourniquet hat assembly set forth in claim 1, wherein said tourniquet fits within a channel positioned about said inner perimeter of said hat.

4. The tourniquet hat assembly set forth in claim 1, wherein said tourniquet is adjustable to increase and decrease said perimeter of said hat, in order to accommodate the size of a wearer's head.

5. The tourniquet hat assembly set forth in claim 1, further including a windlass crank that is removably attached to a bill of said hat, wherein said windlass crank engages with said tightening means in order to tighten said tourniquet.

6. The tourniquet hat assembly set forth in claim 1, further including a windlass crank attached to said tightening member so that a user may grasp and twist said windlass crank to tighten said tourniquet

7. A method for applying a tourniquet, comprising the steps of:
providing a hat with an inner surface;
providing a tourniquet member having a first end and a second end and tightening means; and
removably attaching said tourniquet member to said inner surface of said hat, such that said tourniquet member is positioned about an inner circumference of said hat.

8. The method set forth in claim 7, further including the step of positioning said adjustment member adjacent a rear portion of said hat, so that adjusting said adjustment member serves to adjust the size of said hat.
